(19) United States
(12) Patent Application Publication
(10) Pub. No.: US 2009/0304776 A1
(43) Pub. Date: Dec. 10, 2009

(54) TRANSMUCOSAL DELIVERY OF THERAPEUTIC AGENTS AND METHODS OF USE THEREOF

(76) Inventors: Totada Shantha, McDonough, GA (US); Jessica Shantha, McDonough, GA (US); Erica Shantha, McDonough, GA (US); Lauren Shantha, McDonough, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/339,126

(22) Filed: Dec. 19, 2008

Related U.S. Application Data
(60) Provisional application No. 61/131,241, filed on Jun. 6, 2008.

(51) Int. Cl. A61K 9/70; A61K 9/00; A61K 38/21; A61K 31/79

(52) U.S. Cl. 424/430; 424/400; 424/443; 424/85.5; 424/78.07; 424/446; 424/435; 424/436; 424/434

(57) ABSTRACT

Described herein is a transmucosal delivery device and their use for delivering bioactive agents across a mucosal membrane. The delivery devices contain a pharmaceutically acceptable oxidizing and agents that facilitates the delivery of the blood stream across the mucosal membrane.

### CROSS REFERENCE TO RELATED APPLICATION

This application claims priority upon U.S. provisional application Ser. No. 61/131,241, filed Jun. 6, 2008. This application is hereby incorporated by reference in its entirety for all of its teachings.

### BACKGROUND

More than 21 million people, or 7% of the population, in the United States have diabetes. In addition, there are millions both domestically and abroad who are undiagnosed. In 2005, approximately 1.5 million new cases were diagnosed in people age 20 years or older. The World Health Organization (WHO) recently compiled data that estimates roughly 180 million people have diabetes all over the world and this number is expected to double by 2030.

Traditionally clinicians categorize diabetes into two main groups known as insulin-dependent diabetes mellitus (IDDM) and non-insulin-dependent diabetes mellitus (NIDDM). IDDM is often referred to as Type I diabetes. Type I diabetes, otherwise known as juvenile diabetes, is an autoimmune disease where the immune system attacks insulin producing cells. Thus, cells either do not secrete insulin or secrete an insufficient amount of insulin to maintain adequate blood sugar levels. Unlike Type I diabetes, NIDDM or Type II diabetes occurs mostly in adults. In Type II diabetes, the pancreas makes insulin, but the body's cells do not respond to the circulating insulin due to a resistance to this hormone.

Persons with type II diabetes may not require insulin to survive; however, about 30% benefit from insulin therapy to control the blood glucose. To further complicate diagnoses and treatment, up to 20% of individuals with type II diabetes may in reality have both type I and type II. As type I diabetes progresses, full insulin replacement may be required. This is said to be due to destruction of insulin producing cells by high lipids—a lipocentric theory in its aetiology. (Dixon J B, O'Brien P E, Playfair J; et al. Adjustable gastric banding and conventional therapy for type 2 diabetes: a randomized controlled trial. JAMA. 2008; 299(3):316-323. McGarry J D. What if Minkowski had been ageusic? An alternative angle on diabetes. Science. 1992; 258(5083):766-770. Unger R H. Minireview: weapons of lean body mass destruction: the role of ectopic lipids in the metabolic syndrome. Endocrinology. 2003; 144(12):5159-5165. Unger R H. Reinventing type 2 diabetes: pathogenesis, treatment, and prevention. JAMA. 2008; 299(10):1185-1187. Lee Y, Hirose H, Ohneda M, Johnson J H, McGarry J D, Unger R H. Beta-cell lipotoxicity in the pathogenesis of non-insulin-dependent diabetes mellitus of obese rats: impairment in adipocyte-beta-cell relationships. Proc Natl Acad Sci USA. 1994; 91(23): 10878-10882). Risk factors such as high blood pressure, high cholesterol, genetic predisposition, and aging coupled with obesity and a sedentary lifestyle contribute to this condition.

Many people unknowingly suffer from type II diabetes, and it is rapidly becoming a disease of epidemic proportions for all ages, genders, and ethnicities. In the US alone, this disease costs patients, employers, and insurance companies 174 billion dollars collectively each year. In addition, the number of people worldwide suffering from diabetes rose from 30 million to 230 million over the past two decades. For example, China has the largest number of diabetics in the world over age 20; a total of 39 million people are afflicted with this disease. Likewise, India has the second largest number of diabetics in the world, with about 6 percent of the adult population or an estimated 30 million diabetics. In some Caribbean and Middle Eastern countries, the percentage of diabetic people ranged from 12 to 20 percent. In the world's poorest nations, the disease is a quick death sentence. For example, an insulin dependent person in Mozambique, who requires daily injections of insulin, may not live more than a year, or a person in Mali may not live more than 30 months. However, Americans who receive timely and proper treatment live many years with the disease. As alluded to above, weight gain, excessive carbohydrate intake, and lack of exercise leads to a greater risk of developing Type 2 diabetes. While Type 1 diabetes is responsible for only 5% to 10% of the total reported cases, approximately 90% to 95% of the reported cases are Type 2 diabetes. In either form, diabetes is characterized by high blood sugar levels that result from the body's inability to make insulin or to properly utilized insulin (i.e. insulin resistance). These deficiencies lead to a host of complications including heart disease, cancers, non healing wounds, kidney failure, blindness, stroke, peripheral nerve pain, congenital birth defects, less resistance to infection, and numerous types of heart and blood vessel diseases. Currently, most diabetics fall between the ages of 40 and 59, and millions of diabetics die worldwide every year due to complications or improper treatment. However, these statistics are likely to change in the future, and some estimates indicate that the number of diabetics could grow to 350 million worldwide during the next two decades.

To date, daily insulin injections remain the clinician's preferred treatment of Type 1 and for approximately 20 to 30% of people with Type 2 diabetes. Several alternative forms of insulin such as oral, inhalable, and transdermal exist, but each of these forms has severe drawbacks. For instance, inhalable insulin was linked to various forms of cancer and subsequently withdrawn from the market (see Shantha, T. R., “Unknown health Risks of Inhaled Insulin” Life Extension, pp. 79-82, September 2007; pp 20 Sep. 2008). Various oral insulin formulation medications are highly degraded by stomach acids, and these medications may also cause gastrointestinal and colon cancers. (Shantha T. R. and Jessica G. Shantha; Inhalation Insulin, Oral and Nasal Insulin Sprays for Diabetics: Panacea or Evolving Future Health Disaster Part I; Oral Insulin (swallowed) and Rectal Insulin Suppository for Diabetics: Panacea Or Evolving Future Health Disaster; Part II Townsend Letter December 2008). In addition, transdermal formulations have been cumbersome and difficult to administer on a daily basis. Even injectable insulin has downsides such as injection site discomfort, injection site infection, and difficulty in regulating the proper amount of insulin administered. While current diabetes treatments display numerous shortcomings, a transmucosal insulin delivery system may overcome many of the problems mentioned above, but no such system currently exists. Thus, an important unmet need is to formulate a transmucosal delivery system for insulin while avoiding many of the side effects seen in other treatments.

### SUMMARY

Described herein are transmucosal delivery devices and their use for delivering bioactive agents across a mucosal membrane. The delivery devices contain a pharmaceutically acceptable oxidizing that facilitates the delivery of the bioactive agent to the blood stream across the mucosal membrane. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows a kit composed of (a) a pharmaceutically acceptable oxidizing agent patch and (b) a bioactive agent patch.

FIG. 2 shows a multilayered patch having a removable sleeve.

FIG. 3 shows a multilayered patch having a removable sleeve and an impermeable substrate.

FIG. 4 shows a multilayered patch having two removable sleeves, where the bioactive layer is sandwiched between two oxidant layers.

FIG. 5 shows the top view of a patch having a bioactive agent on the surface of an impermeable patch with oxidizer spots on the surface of the bioactive agent.

FIG. 6 shows the cross-sectional view of a patch having a bioactive layer on the surface of an impermeable patch with oxidizer spots on the surface of the bioactive layer.

FIG. 7 shows the cross-sectional view of an impermeable patch with an indentation for receiving a bioactive layer or agent.

FIG. 8 shows the cross-sectional view of a patch having a bioactive layer on each side of an impermeable patch with oxidizer spots on the surface of the bioactive layer.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a peptide” includes mixtures of two or more such peptides, and the like.

“Optional” or “optionally” means that the subsequently described event or circumstance can or cannot occur, and that the description includes instances where the event or circumstance occurs and instances where it does not. For example, the phrase “optional mucosal penetration enhancer” means that the bioactive agent can or can not be included.

Described herein is a transmucosal delivery device and methods of use thereof. In general, the delivery device provides an efficient method for delivering a bioactive therapeutic, a pharmacological, a biologic, and a nutriceutical agent across a mucosal membrane. Transmucosal delivery of bioactive therapeutics offers several advantages over oral delivery, parenteral injection, transdermal delivery, and inhalation methods. These advantages are avoidance of first pass metabolism in the liver, easily discontinuing administration of such bioactive therapeutics, constant maintenance of the bioactive agent in the blood for potentially longer periods of time, and lower applicable doses when compared to oral and inhalable treatments.

A pharmaceutically acceptable oxidizing agent is also incorporated into the delivery device mentioned above, which facilitates the delivery of the bioactive agent across mucosal membrane. The function of the pharmaceutically acceptable oxidizing agent is described in more detail below. In certain aspects, the bioactive agent and the pharmaceutically acceptable oxidizing agent are in close proximity to one another such that when the delivery device is applied to the mucosal membrane, the bioactive agent and the pharmaceutically acceptable oxidizing agent are delivered concurrently to the subject.

The delivery device can be composed of a variety of different biocompatible materials that acts as a substrate for the bioactive agent and the pharmaceutically acceptable oxidizing agent. The bioactive agent and the pharmaceutically acceptable oxidizing agent can be applied to the material using techniques known in the art. For example, a membrane composed of the biocompatible material may be treated or sprayed with the bioactive agent, a pharmaceutically acceptable oxidant, or combination thereof. In other aspects, the biocompatible membrane can be formed by extruding a biocompatible material and the bioactive agent, a pharmaceutically acceptable oxidant, or combination such that the bioactive agent and/or pharmaceutically acceptable oxidant are dispersed throughout the membrane. In other aspects, the device may include multiple layers or laminates of bioactive agent and pharmaceutically acceptable oxidizing agents.

In one aspect, the biocompatible material forms a network of polymer chains that are water-insoluble as found in a colloidal gel in which water is the dispersion medium. In other aspects, the biocompatible material is also biodegradable, where the material disintegrates over time. The biocompatible material generally provides a degree of flexibility to the device similar to natural tissues. For example, the biocompatible material can be a gel or hydrogel that swells when contacted with water or other body fluids. Examples of such materials include, but are not limited to, cationic polymers, acrylic polymers, cellulose polymers, polyurethane, polylactic/polyglyocolic acids, polyamino acids, polysaccharides, polyureas, polyvinyl alcohols, polyvinylpyrrolidone, natural proteins, or any combination thereof. As will be described below, in certain aspects, the device may have multiple, individual internal layers, where each layer can produced from the same or different biocompatible material. The selection of the biocompatible material can be based upon several factors including the permeability and diffusion rates of the material, the thickness of the material, and the particular mucosal membrane the device will be applied to. For example, the device permits the diffusion of the bioactive agent and the pharmaceutically acceptable oxidizing agent. The dose of the bioactive agent may be the appropriately indicated therapeutic dose, or the dose of the bioactive agent within the patch may vary from 5 to 30 International Units.

A variety of different bioactive agents can be delivered using the devices described herein. In one aspect, the bioactive agent can be a natural or synthetic oligonucleotide, a natural or modified/blocked nucleotide/nucleoside, a nucleic acid, an antibody or fragment thereof, a hapten, a biological ligand, a virus, a lipid membrane, nicotine, hormones, anti-cytokine factors; anti-angiogenesis agents, anti-cancer drugs, nutriceuticals, a polysaccharide protein lipid complex, monoclonal antibodies, multi-clonal antibodies, or antigens or a small pharmaceutical molecule.

In one aspect, the bioactive agent can be a protein or peptide. For example, the protein can include peptides, fragments of proteins or peptides, membrane-bound proteins, or nuclear proteins. The protein can be of any length, and can include one or more amino acids or variants thereof. The protein(s) can be fragmented, such as by protease digestion, prior to analysis. A protein sample to be analyzed can also be subjected to fractionation or separation to reduce the complexity of the samples. Fragmentation and fractionation can also be used together in the same assay. Such fragmentation and fractionation can simplify and extend the analysis of the proteins. In one aspect, the bioactive agent can be incorporated into nano particles for easy absorption from the mucosa.

Many peptides and proteins have prophylactic and therapeutic uses. For example, clinicians use insulin to lower blood sugar and to treat diabetes. Likewise, human growth hormone may be used to treat dwarfism or for hormone replacement therapy in middle-aged persons. In addition, vasopressin and vasopressin analogues are used therapeutically to treat von Willebrand disease, extreme cases of bedwetting by children, and other conditions which require vasoconstrictive properties. In one aspect, the transmucosal delivery device includes a protein or peptide such as insulin, calcitonin, vasopressin, luteinizing hormone-releasing hormone, leuprolide, thyrotropin-releasing hormone, cholecystokinin, alpha-interferon, beta-interferon, gamma-interferon, human growth hormone, alpha-1-transforming growth factor, beta-1-transforming growth factor, granulocyte colony stimulating factor (G-CSF), granulocyte macrophage colony stimulating factor (G-MCSF), parathyroid hormone (PUT), glucagons, somatostatin, vaso-active intestinal peptide (VIP), or any combination thereof.

Dosing with respect to the amount of bioactive agent is dependent on the type, severity, and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates. For example, insulin may be used a bioactive agent, and the insulin can be short or long acting and dosing may include 3, 5, 10, 15, 20, 30 International Units per patch or delivery system.

As described above, the pharmaceutically acceptable oxidizing agent facilitates the delivery of the bioactive agent through the mucosal membrane. In general, the oxidizing agent can react with molecules present in the mucosal membrane that would adversely react with the bioactive agent. For example, reduced glutathione can inactivate bioactive agents by breaking crucial molecular bonds. Not wishing to be bound by theory, when delivering insulin either transmucosally or transdermally, reduced glutathione can inactivate insulin. Specifically, insulin has numerous disulfide bonds which are crucial for its protein conformation, biological activity, and subsequent therapeutic effects. Reduced glutathione will inactivate insulin by reducing or breaking insulin's disulfide bonds. Once these disulfide bonds are broken, insulin becomes inactive due to lost protein conformation and biological activity. Thus, the administration of the oxidant or oxidizing agents using the devices described herein prevents the inactivation of the bioactive agent. Specifically, applying an oxidant or a pharmaceutically oxidizing agent transmucosally will lower or prevent the effects reduced proteins and reduced biological molecules have on the bioactive agents. In this manner, the inactivation of bioactive agents via reduction or cleavage of crucial molecular bonds will be avoided. The selection and amount of the pharmaceutically acceptable oxidizing agent can vary depending upon the bioactive agent that is to be administered. In one aspect, the oxidizing agent includes, but is not limited to, iodine, povidone-iodine, any source of iodine or combinations of oxidants, silver protein, active oxygen, potassium permanganate, hydrogen peroxide, sulfonamides, dimethyl sulfoxide or any combination thereof. These oxidizing agents may also act as absorption agents which help facilitate delivery of a therapeutic agent onto and into a mucosal membrane. In one aspect, the oxidant is at least greater than 1% weight per volume, weight per weight, or mole percent. In another aspect, the mucosal membrane permeability enhancer may be at least greater than 1.5%, 2.0%, 2.5%, 3.0%, 3.5%, 4.0%, or 4.5% weight per volume, weight per weight, or mole percent. In this aspect, the oxidant may range from 2% to 10%, 2% to 9.5%, 3% to 8%, 3% to 7%, or 4% to 6% weight per volume, weight per weight, or by mole percent.

Additional components can be present in the device to facilitate the delivery of the bioactive agent mucosally to the subject. In one aspect, transmucosal penetration enhancers can be used to further expedite the entry of the bioactive agent into the mucosa and ultimately the blood stream. Penetration enhancers work by increasing permeability across a particular boundary or membrane. Penetration enhancers not only penetrate a membrane efficiently, but these enhancers also enable other bioactive agents to cross a particular membrane more efficiently. Penetration enhancers produce their effect by various modalities such as disrupting the cellular layers of mucosa, interacting with intracellular proteins and lipids, or improving partitioning of bioactive agents as they come into contact with the mucosal membranes. With these enhancers, macromolecules up to 10 kDa are able to pass through the mucosal membrane.

These enhancers should be non-toxic, pharmacologically inert, non-allergic substances. In general these enhancers may include anionic surfactants, ureas, fatty acids, fatty alcohols, terpenes, cationic surfactants, nonionic surfactants, zwitterionic surfactants, polyols, amides, lactam, acetone, alcohols, and sugars. In one aspect, the penetration enhancer includes dialkyl sulfoxides such as dimethyl sulfoxide (DMSO), decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof. In another aspect, the penetration enhancer may include lauryl alcohol, diisopropyl sebacate, oleyl alcohol, diethyl sebacate, dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethyl-hexyl pelargonate, butyl benzoate, benzyl benzoate, benzyl salicylate, dibutyl phthalate, or any combination thereof. In one aspect, the mucosal membrane permeability enhancer is at least greater than 1% weight per volume, weight per weight, or mole percent. In another aspect, the mucosal membrane permeability enhancer may be at least greater than 1.5%, 2.0%, 2.5%, 3.0%, 3.5%, 4.0%, 4.5% up to 50% weight per volume, weight per weight, or mole percent. In one aspect, the mucosal membrane permeability enhancer is dimethyl sulfoxide. In this aspect, the amount of dimethyl sulfoxide may range from 2% to 10%, 2% to 9.5%, 3% to 8%, 3% to 7%, or 4% to 6% weight per volume, weight per weight, by mole percent, or any effective therapeutic amount.

In other aspects, these additional components may include antiseptics, antibiotics, anti-virals, anti-fungals, anti-inflammatories, anti-dolorosa, antihistamines, steroids, and vasoconstrictors within the device to reduce inflammation, irritation, or reduce rapid absorption by blood vessels on and around the mucosal membrane. Such vasoconstrictors may include phenylephrine, ephedrine sulfate, epinephrine, naphazoline, neosynephrine, vasoxyl, oxymetazoline, or any combination thereof. Such anti-inflammatories may include non-steroidal anti-inflammatory drugs (NSAIDs). NSAIDs alleviate pain and inflammation by counteracting cyclooxygenase and preventing the synthesis of prostaglandins. In one aspect, NSAIDs include celecoxib, meloxicam, nabumetone, piroxicam, naproxen, oxaprozin, rofecoxib, sulindac, ketoprofen, valdecoxid, anti-tumor necrosis factors, anti-cytokines, anti-inflammatory pain causing bradykinins or any combination thereof. Such antiseptics, anti-virals, anti-fungals, and antibiotics, may include ethanol, propanol, isopropanol, or any combination thereof; a quaternary ammonium compounds including, but not limited to, benzalkonium chloride, cetyl trimethylammonium bromide, cetylpyridinium chloride, benzethonium chloride, or any combination thereof; boric acid; chlorhexidine gluconate, hydrogen peroxide, iodine, mercurochrome, ocetnidine dihydrochloride, sodium chloride, sodium hypochlorite, silver nitrate, colloidal silver, mupirocin, erthromycin, clindamycin, gentamicin, polymyxin, bacitracin, silver, sulfadiazine, or any combination thereof.

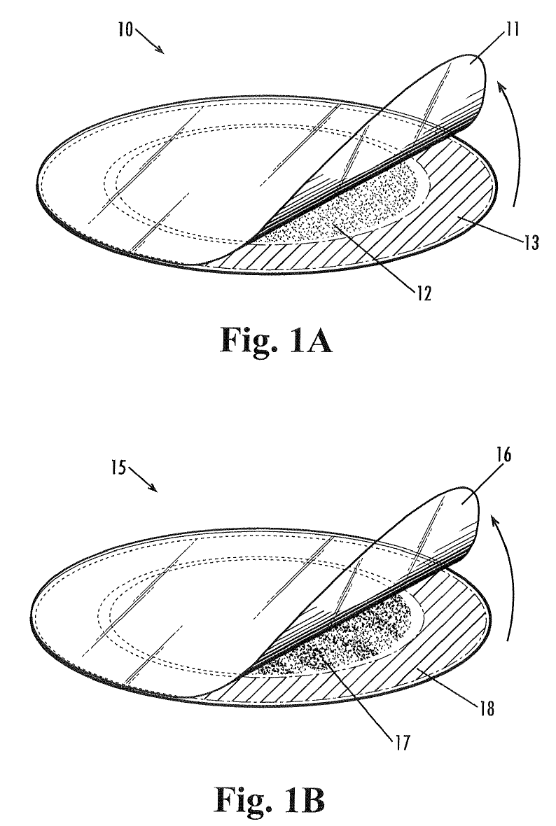

FIGS. 1-8 show examples of several different delivery devices described herein. FIG. 1 shows a two patch system. Referring to FIG. 1A, the oxidant patch 10 has a removable protective cover 11 that can be readily peeled from the oxidant patch. Upon removal of the cover, the oxidant region 12 present on patch substrate 13 is exposed. The patch substrate can be composed of an impermeable material so that the oxidant does not diffuse away from the mucosal membrane when applied to the mucosa. The oxidant patch is applied to the mucosal membrane such that the oxidant comes into contact with the mucosal membrane. The oxidant patch is removed after a sufficient time to allow the pharmaceutically acceptable oxidizing agent to diffuse from the patch onto the mucosal membrane. After removal of the oxidant patch, a bioactive patch 15 (FIG. 1B) can be applied to the region of the mucosa where the oxidant patch was previously applied. The structure of the bioactive patch 15 can be similar to if not identical to that of the oxidant patch 10, where upon removal of the protective cover 16 bioactive region 17 is exposed on patch substrate 18. The bioactive patch is applied to the mucosa for a sufficient time to ensure that most if not all of the bioactive agent has diffused from the patch into the mucosal membrane. In this aspect, the oxidant patch 10 and bioactive patch can be used as a kit.

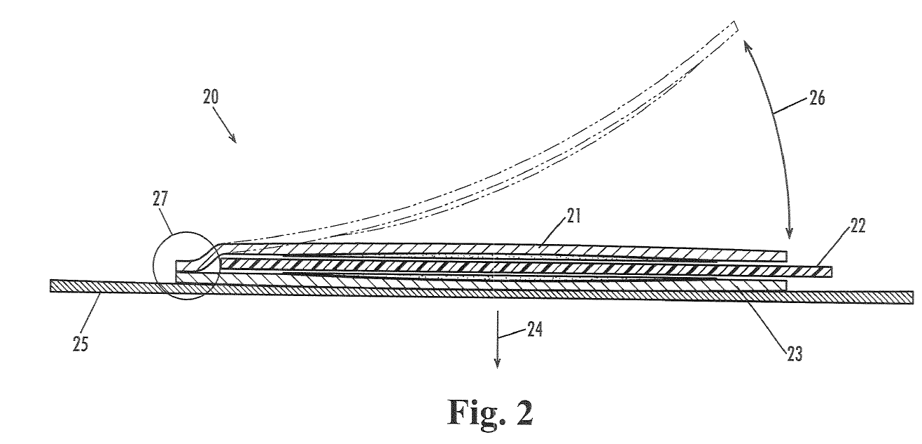

FIG. 2 depicts another device described herein, where the device 20 has been applied to mucosal membrane 25. Device 20 is composed of a bioactive membrane 21, a removable sleeve 22, and oxidant membrane 23. The bioactive membrane and oxidant membrane can be produced using the components described above using techniques known in the art. The dimensions of the membranes can vary depending upon the application of the device. The components and dimensions of the bioactive membrane and oxidant membrane are selected such that the bioactive agent and oxidant can readily diffuse from and permeate through the membranes and come into contact with the mucosal membrane. The removable sleeve can be produced from any inert substance that is resistant to tearing upon removal. In this aspect, the device 20 is operated by lifting the bioactive membrane (depicted as arrow 26) followed by removal of sleeve 22. The bioactive membrane is then folded back on the oxidant membrane 23. The bioactive membrane and oxidant membrane can be adhered to one another (27 in FIG. 2) by a biocompatible adhesive. Alternatively, the ends of the bioactive membrane and oxidant membrane can be melt-pressed to one another using techniques known in the art. The device 20 may optionally have a protective layer applied to the outer surface of the oxidizing membrane to prevent any premature release of the oxidizing agent. The protective layer can be composed of any impermeable and durable material. After removal of the sleeve 22 and optional protective cover, the device 20 can be applied to the mucosal membrane of choice.

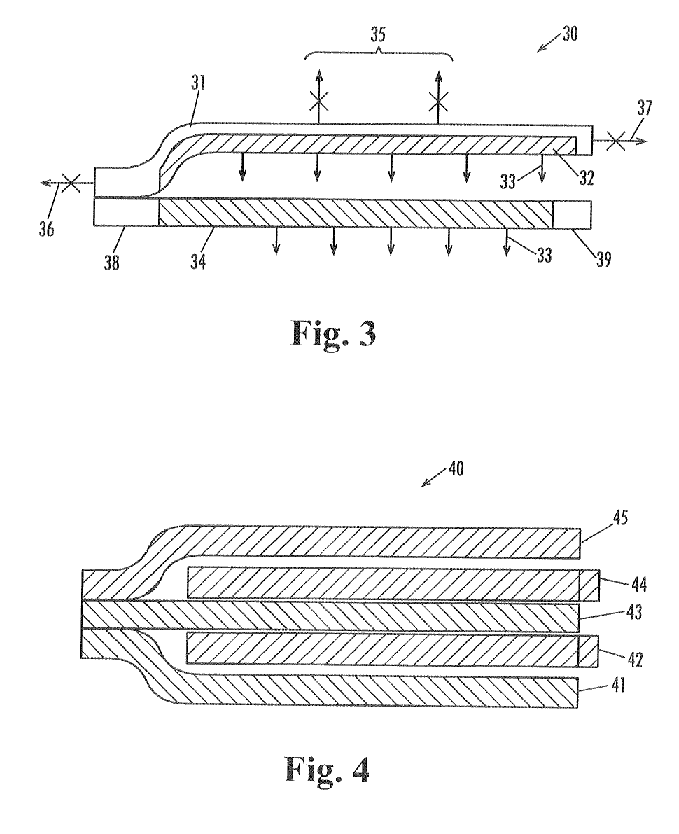

FIG. 3 depicts another feature of the device as shown in FIG. 2 with the sleeve removed. Referring to FIG. 3, the device 30 has an impermeable layer 31 that surrounds the bioactive layer 32 so that the bioactive agent present in bioactive layer 32 diffuses downward (depicted as 33 in FIG. 3) and towards the oxidant layer 34. The impermeable layer prevents the diffusion of bioactive agent from away from the oxidant layer (35) or from the sides of the device (36 and 37). The edges of the oxidant layer 34 (38 and 39) are also surrounded by the same or different impermeable layer 31. Thus, the oxidant and bioactive agent are delivered to a specific region of the mucosal membrane, which enhances the efficiency of the delivery device.

FIG. 4 depicts another aspect related to the embodiment in FIG. 2. Referring to FIG. 4, device 40 is composed of two removable sleeves (42 and 44) and two oxidant membranes (41 and 45). Upon removal of sleeves 42 and 44 in a manner similar to that described above in FIG. 2, a three-layered device is produced, where the bioactive membrane 43 is sandwiched between two oxidant layers. This aspect is useful when the mucosal membrane surrounds the device (e.g., vaginal or rectal applications).

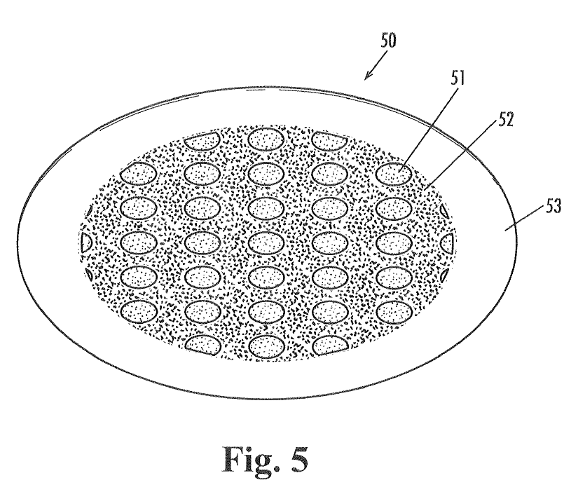

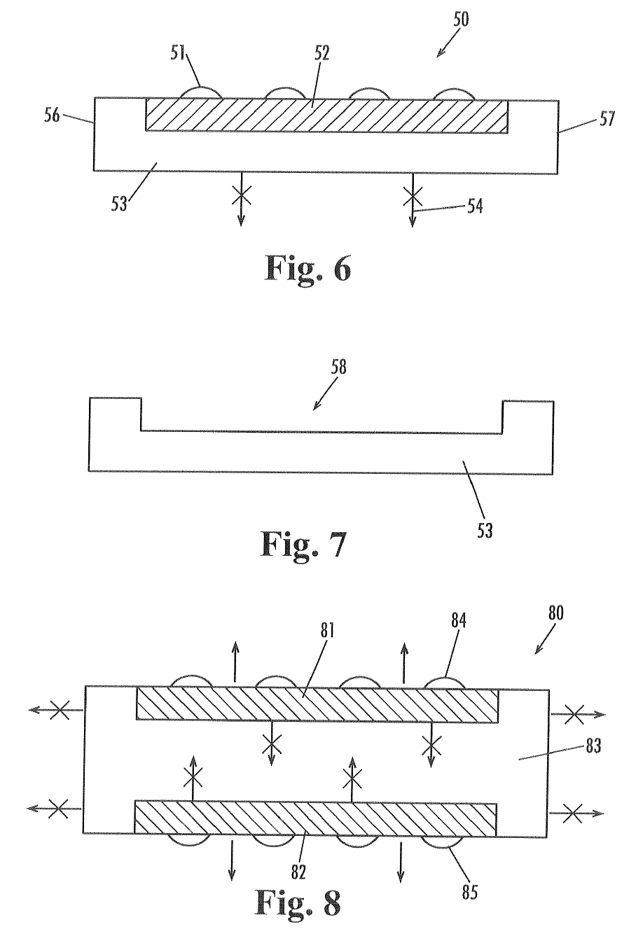

Another device as described herein is depicted in FIGS. 5 and 6. FIGS. 5 and 6 show the top view and cross-sectional views of patch 50, respectively. The patch is composed of a plurality of oxidizer spots 51, containing a pharmaceutically acceptable oxidizing agent, on the surface of a bioactive layer 52. The bioactive layer 52 is adjacent to surface of the patch substrate 53. The patch substrate is generally composed of an impermeable material as described above such that the bioactive agent and oxidant do not diffuse through the underside (54) of the patch. The bioactive layer and oxidizer spots can be applied to the patch substrate using techniques known in the art. With respect to the bioactive agent, it can be applied directly to the patch substrate in a desired pattern. Alternatively, the bioactive agent can be admixed with a biocompatible polymer as described above followed by applying the admixture to the surface of the patch substrate to produce the bioactive layer. In one aspect, referring to FIG. 6, the patch substrate 53 can receive the bioactive layer 52 such that the bioactive agent does not permeate and release from the sides 56 and 57 of the patch substrate 53. In this aspect, the patch substrate 53 has an indentation 58 for receiving the bioactive layer (FIG. 7). Once the bioactive layer has been applied to the patch substrate, the oxidizer spots can be applied to the bioactive layer. Although depicted as spots, the oxidant can be applied in a variety of different shapes and sizes on the bioactive layer. When ready to use device 50, the side of the device with the oxidizing spots is applied directly to the mucosal membrane.

FIG. 8 shows a related device as depicted in FIGS. 5 and 6. FIG. 8 shows a cross-sectional view of bi-layered patch 80. Referring to FIG. 8, the bioactive layers 81 and 82 are adjacent to patch substrate 83, with oxidizing spots 84 and 85 adjacent to the bioactive layers. The patch substrate 83 contains the bioactive layer so that the bioactive agent does not diffuse from the sides of the patch but directly to a specific region of the mucosal. Similar to above, optional protective layers can be applied on top of the oxidant and bioactive layer to prevent release of these materials from the patch.

Although the delivery devices depicted in the figures are patches, it is contemplated that the delivery device can assume other shapes and materials. For example, the delivery device may also include a capsule. This capsule may be oval, cubed, square, or rectangular shaped with either smooth, rounded edges or rigid, blunt edges. This capsule is adequately shaped to permit positioning between the mucous membranes, and teeth with gums. In one aspect, the capsule has a core composed of a bioactive agent and a second layer surrounding the core composed of the pharmaceutically acceptable oxidizing. In other aspects, the capsule may contain a heterogeneous mixture of the bioactive agent and the pharmaceutically acceptable oxidizing agent. The capsules can be formulated to control the rate of release of the bioactive agent and the pharmaceutically acceptable oxidizing agent. In other aspects, the device can be a gel, an ointment, a solution, a paste, or a powder, where the bioactive agent and pharmaceutically acceptable oxidizing agent are formulated with a pharmaceutically acceptable carrier. In other aspects, the bioactive agent and the pharmaceutically acceptable oxidizing agent can be applied to an absorbent gauze.

The delivery devices described herein can be applied to any mucosal membrane present in the subject. The mucosal membrane as used herein also includes the submucosal membrane component with its blood vessels and neuronal components. For example, the mucosal membrane may be located within the nasal cavity, the mouth, the gums, the lips, the cheeks, or underneath or on top of the tongue. Alternatively, the mucus membrane may be located in the rectum, vagina, or the eyes. As described above, the delivery device facilitates the entry of the bioactive agent into the mucosal membrane and ultimately the circulatory system of the subject, where the bioactive agent will be effectively delivered to the target cells and organs.

The delivery devices described herein can be useful in treating or preventing numerous diseases. In one aspect, the devices described herein can be used to treat the symptoms of diabetes. The term “treat” as used herein includes reducing one or more symptoms of the disease or maintaining the current symptom(s) such the symptom does not worsen. For example, when “treating” diabetes, blood sugar levels must be maintained at physiological levels. This treatment may entail administration of insulin. As described above, Type I and Type II diabetes are marked by high blood sugar levels that result from the body's inability to make or use insulin. If left untreated, diabetes' debilitating effects may lead to blindness, stroke, and ultimately death. In one aspect, a pharmaceutically acceptable oxidizing agent such as iodine can be applied to a mucosal membrane to counteract any effect reduced proteins or biological molecules may have on insulin. Thus, in one aspect, the devices described herein provide an effective way to deliver insulin to patients suffering from diabetes. In this aspect, the device can be applied to a mucosal membrane located on the gums, lips, or cheek inside the mouth cavity or a mucosal membrane within the nasal cavity. Thus, by effectively delivering insulin mucosally to a subject, the symptoms of diabetes can be alleviated.

To further enhance transmucosal delivery, sonophoresis may be used. Sonophoresis is a process that exponentially increases the absorption of bioactive agents into the mucosal and submucosal layers. In sonophoresis, ultrasound waves stimulate micro-vibrations within the mucosal and submucosal layers that can facilitate the uptake of bioactive agents. In one aspect, sonophoresis may be applied close to the mucosa either before or after applying the oxidizing agent. Sonophoresis increases transmucosal and submucosal penetration and absorption of various bioactive agents including peptides and proteins such as, for example, insulin, calcitonin, vasopressin, luteinizing hormone-releasing hormone, leuprolide, thyrotropin-releasing hormone, cholecystokinin or any combination thereof.

Like sonophoresis, iontophoresis, electrophoresis, fractional laser, or mechanical force such as magnetic force, vibration, vibroacoustic force may be used to expedite absorption of bioactive agents into the mucosal and submucosal layers. Iontophoresis and electrophoresis are non-invasive methods for propelling high concentrations of a charged substance such as protein, peptide, or bioactive-agent through a membrane by using a low electrical current. In one aspect, iontophoresis may be applied to the mucosal membrane or skin to further facilitate absorption of the oxidizing agent and bioactive agent. In another aspect, ultrasound may be used at low frequencies to facilitate the absorption of the oxidizing agent and bioactive agent. For example, less than 2.5 MHz may be used, less than 2.0 MHz may be used, less than 1.5 MHz, or less than 1.0 MHz may be used. In a further aspect, when vibroacoustics are used, specified pulse profiles are employed. These vibroacoustics may include sequences of pulses ranging from one-half second to three seconds, modulated with an oscillatory signal a frequency ranging from 1 Hz to about 1500 Hz, and having a pulse amplitude in the range from about 20 to 5000 microns. In yet another aspect, mechanical delivery forces such as sonophoretic or vibroacoustic treatments may be applied to the mucosal membrane or skin either before or after the application of the transmucosal delivery device. When applied on the surface of the skin, ultrasound vibration (vibroacoustic) mechanical forces can be transmitted to the mucous membrane of the vestibule of the mouth which will enhance the absorption of the bioactive agents including insulin into the blood stream. Bioactive agents such as, for example, insulin, calcitonin, vasopressin, luteinizing hormone-releasing hormone, leuprolide, thyrotropin-releasing hormone, anti-inflammatory cytokines such as anti-tumor necrosis factors, cholecystokinin, chemotherapeutic agents against infections and cancers, any and all therapeutic pharmacological, biologic, and nutriceuticals agents can undergo iontophoresis.

Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed:

1. A transmucosal delivery device comprising a first layer and a second layer, wherein the first layer comprises a bioactive agent and the second layer comprises a pharmaceutically acceptable oxidizing agent.

2. The device of claim 1, wherein the device comprises a patch having a first surface and second surface, wherein the first layer is adjacent to the first surface of the patch, and the second layer is adjacent to the first layer.

3. The device of claim 2, wherein an additional first layer is adjacent to the second surface of the patch, and an additional second layer is adjacent to the additional first layer.

4. The device of claim 2, wherein the patch comprises a substantially impermeable material.

5. The device of claim 1, wherein the first layer and the second layer comprise a gel, a hydrogel, an acrylic polymer, a cellulose polymer, a polyurethane, a polylactic/polyglycolic acid, a polyamino acid, a polysaccharide, a polyurea, a polyvinyl alcohol, a polyvinylpyrrolidone (povidone), or any combination thereof.

6. The device of claim 1, wherein the first layer and second layer are separated by a removable separating layer.

7. The device of claim 1, further comprising a third layer comprising a pharmaceutically acceptable oxidizing agent, wherein the first layer is between the second layer and third layer.

8. The device of claim 7, wherein the first layer is adjacent to the second layer and third layer.

9. The device of claim 7, wherein the first layer and second layer are separated by a first removable separating layer, and the first layer and third layer are separated by a second removable separating layer.

10. The device of claim 1, wherein the first layer and second layer comprises a biocompatible polymer.

11. The device of claim 1, wherein the device comprises a capsule, and the capsule comprises a core comprising the first layer and a second layer adjacent to the core.

12. The device of claim 1, wherein the bioactive agent comprises a peptide or a protein.

13. The device of claim 1, wherein the bioactive agent comprises a peptide or protein, and the peptide or protein comprises insulin, calcitonin, vasopressin, luteinizing hormone-releasing hormone, leuprolide, thyrotropin-releasing hormone, cholecystokinin, alpha-interferon, beta-interferon, gamma-interferon, human growth hormone, alpha-1-transforming growth factor, beta-1-transforming growth factor, granulocyte colony stimulating factor (G-CSF), granulocyte macrophage colony stimulating factor (G-MCSF), parathyroid hormone (PUT), glucagons, somatostatin, vase-active intestinal peptide (VIP); anti-cytokines, nutriceutical agents or any combination thereof.

14. The device of claim 1, wherein the pharmaceutically acceptable oxidizing agent comprises iodine, povidone-iodine, any source of iodine or combinations of oxidants, silver protein, active oxygen, potassium permanganate, sulfonamides, or any combination thereof.

15. The device of claim 1, further comprising a mucosal penetration enhancer incorporated into the first layer, the second layer, or any combination thereof.

16. The device of claim 15, wherein the mucosal penetration enhancer comprises dimethyl sulfoxide, anionic surfactants, urea's, fatty acids, fatty alcohols, terpenes, cationic surfactants, nonionic surfactants, zwitterionic surfactants, polyols, amides, lactam, acetone, alcohols, sugars or any combination thereof.

17. The device of claim 1, wherein the amount of bioactive agent is from 1% to 95% and the amount of pharmaceutically acceptable oxidizing agent is from 1 to 90%.

18. A pharmaceutical composition comprising a bioactive agent, a pharmaceutically acceptable oxidizing agent, and a pharmaceutically acceptable carrier.

19. The composition of claim 18, wherein the pharmaceutically acceptable carrier comprises a gel, a hydrogel, an ointment, a solution, a paste, a powder, an absorbent gauze, a spray, a patch, a capsule or any combination thereof.

20. The composition of claim 18, wherein the bioactive agent comprises insulin and the pharmaceutically acceptable oxidizing agent comprises iodine.

21. A kit comprising (a) comprising a bioactive agent on a first pharmaceutically acceptable carrier and (b) a pharmaceutically acceptable oxidizing agent on a second pharmaceutically acceptable carrier.

22. A method of delivering a bioactive agent to a subject comprising at least one mucosal membrane, the method comprising applying the delivery device of claim 1 to the mucosal membrane.

23. The method of claim 22, wherein the mucosal membrane is located within the nasal cavity, the mouth, gums, lips, cheeks, underneath the tongue, the rectum, or the vagina.

24. The method of claim 22, further comprising applying sonophoresis, iontophoresis, electrophoresis, electroosmosis, vibroacoustic force, or a combination thereof to the mucosal membrane either before or after contacting the mucosal membrane with the delivery device.

25. A method of delivering a bioactive agent to a subject comprising at least one mucosal membrane using the kit of claim 21, the method comprising contacting the mucosal membrane with (1) a pharmaceutically acceptable oxidizing agent on a second pharmaceutically acceptable carrier followed by (2) contacting the mucosal membrane with the bioactive agent on a first pharmaceutically acceptable carrier.

26. A method for treating a subject with diabetes comprising applying the delivery device of claim 1 to at least one mucosal membrane in the subject, wherein the bioactive agent comprises insulin and the pharmaceutically acceptable oxidizing agent comprises iodine.

27. The method of claim 26, wherein the device further comprises a mucosal penetration enhancer comprising dimethyl sulfoxide, anionic surfactants, ureas, fatty acids, fatty alcohols, terpenes, cationic surfactants, nonionic surfactants, zwitterionic surfactants, polyols, amides, lactam, acetone, alcohols, sugars, or any combination thereof in the first layer, second layer, or both first and second layer.
